(19) United States
(12) Patent Application Publication
(10) Pub. No.: US 2023/0200971 A1
(43) Pub. Date: Jun. 29, 2023

(54) A BODY OF THE DEVICE TO POSITION A CORONARY STENT IN CORONARY ARTERIES OR PULL A CATHETER TRANSDUCER FOR INTRA VASCULAR ULTRASOUND

(71) Applicant: SEVEN SONS LTD, Tel Aviv (RU)

(72) Inventor: Vladimir Borisovich TRAPEZNIKOV, Moscow (RU)

(73) Assignee: SEVEN SONS LTD, Tel Aviv (RU)

(21) Appl. No.: 18/010,150

(22) PCT Filed: Dec. 7, 2020
(86) PCT No.: PCT/RU2020/000666

§ 371 Date: Dec. 13, 2022

(30) Foreign Application Priority Data
Jun. 30, 2020 (RU) 2020121738

(51) Int. Cl. A61F 2/01; A61B 8/12

(52) U.S. Cl. CPC A61F 2/011; A61B 8/12; A61F 2250/0093

(57) ABSTRACT

A body of the device to position a coronary stent or pull a catheter transducer for intravascular ultrasound is composed of a front and a rear portion each of which comprises two similar halves, a ring on the front smooth portion of the body and a brace over the rear portion of the rear portion of the body. The rear portion of the body overlaps the front portion of the body. The front portion of the body is truncated on one side thereof and continuously extends on the opposite side thereof in the form of a smooth cylindrical portion of a smaller diameter and a runner guide. A cylindrical slot with point elements is provided along the circumference of the cylindrical portion of the front portion of the body on the side of the runner guide. A cylindrical brace is mounted on the rear portion of the body.

This invention relates to a body of the device suitable for use in medicine to position a coronary stent in the most accurate, fast and safe manner on a delivery system to carry out endovascular coronary stenting or pull a catheter transducer for intravascular ultrasound (IVUS).

When the device is used to pull the catheter transducer, such catheter transducer is installed in the device instead of the coronary stent delivery system in much the same manner.

For intravascular imaging, the guiding catheter is suitably installed within the coronary ostium so that the guiding catheter can be pulled through the target narrowing to distal arteries. The device is placed on the digital IVU Scatheter in the same manner as it normally placed on the coronary stent delivery system A mechanical transducer of the Revolution (Volcano (Philips)) type or other similar devices cannot be currently used due to specific features of their pulling mechanisms. The device is positioned as close as possible to the proximal section of the transmitting catheter, i.e., the section where the transmitting catheter is connected by means of a wire to a station or built-in interface. The transducer is moved through the guide to a distance 30-40 more distal than the target segment. When in the appropriate zone, the device is fixed on the digital IVUS catheter whereupon the device body is rotated to slowly move the device using the procedure similar to the stent positioning procedure for high-precision estimation of this section.

Known on the medical equipment market are devices proposed by the same applicant to position stents in the coronary arteries (RU2598798, RU2652732, RU2650038, RU2661092, RU2710216), with the bodies of such devices differing from the proposed invention the advantages of which are as follows:
easy and short-time assembly of the device body owing to a brace and a ring and because there is no need for glue to mate the parts;
higher strength of the product owing to the brace and a large number of tapered projections and their mating tapered holes in the front portion of the device body;
greater rigidity of the front portion of the device body when a retainer operates, such rigidity provided by a thicker wall of the front truncated portion of the body in the retainer lug zone, with the maximum possible number of the tapered projections and mating taperedholes made along the edges of the thick wall;
no need for a tube to position a coronary stent on the delivery system inside the device, such positioning provided by a fillet inside the front truncated portion of the body and chamfers on the rear end portion of the body and a runner in their central hole;
improved performance of the product during coronary stent positioning owing to greater rigidity of the runner guides provided by an additional wall interconnecting the guides;
no need for gluing and enhanced mating force of the guides for the runner of the front portion of the body owing to transverse walls provided at the end of the guides, with a tapered projection and mating tapered hole;
possibility of use of the device body to pull the catheter transducer;
greater rigidity of coupling of the halves of the rear portion of the body owing to additional longitudinal slots and longitudinal projections along the edge of the internal side of the rearportion of the body in the overlap zone;
maintenance of integrity of the device body in the course of operation owing to the brace securely retained by the projections at the end of the rear portion of the device body;
when the coronary stent is being positioned or catheter transducer is being pulled, the rear portion of the device body can be readily rotated with the aid of a ring owing to parts engagement;
when the coronary stent is being positioned, the brace cannot be displaced around the rear portion of the body owing to the longitudinal projections on the external side of the rear portion of the body in the internal thread zone and internal guides on the brace;
no need for accurate positioning of the brace with respect to the rear portion of the body when the body of the device is being assembled; simple and fast assembly owing to specially shaped longitudinal projections on the external side of the rear portion of the device body in the internal thread zone;
reduced load to the operator's fingers to close the retainer owing to a wall directed at an angle to a point wall projection provided in the opening for the retainer lugs and designed to hold the retainer lugs in the extreme position.

The claimed invention is industrially applicable.

The subject matter of the present invention is a body of the device to position a coronary stent on a delivery system in coronary arteries or pull a catheter transducer for intravascular ultrasound.

The body is composed of a retainer with a thickening with lugs, a runner with a hole to accommodate a delivery system with projections designed to engage an internal double-start thread of a rear cylindrical portion of the body and with projections used for movement along guides inside the body on one side, and a cap with a hole to accommodate a delivery system with an elastic bushing with a hole to position the delivery system inside with a cone-shaped and end surfaces corresponding to similar surfaces inside the cap and at the end of the runner, installed in the threaded cap to be screwed on the runner on the other side.

A mechanism housed in the body is a subject matter of an independent patent (Patent RU 2661092) of the same applicant and cited herein solely for illustration and better understanding of the claimed invention.

The device body is composed of two components one of which is essentially a part comprising a front portion truncated on one side thereof and a cylindrical portion on the opposite side thereof, a smooth cylindrical portion of a smaller diameter and guides for a runner, said component forming the front portion of the body, and the other component includes a part arranged over the smooth cylindrical portion of the first component and a rear part with the internal double-start thread and a chamfered end face, said component forming the rear portion of the body.

The front portion of the body is truncated on the front side thereof and continuously extends on the opposite side thereof in the form of a cylindrical portion, smooth cylindrical portion of a smaller diameter and runner guides interconnected by longitudinal walls and provided at the end with transversal walls having tapered holes and mating tapered projections to couple the body halves.

The front truncated portion of the body is adapted to be held by the fingers of the surgeon's left hand and provided to this end with recesses allowing the surgeon to fix the device in the desired position, with the recesses also serving as a pad to fix a coronary catheter. For more convenient arrangement of the coronary stent delivery system and catheter transducer, the walls in the front truncated portion of the body are made rounded on the internal side opposite to the recesses for the operator's left hand fingers where said walls contact the mouth of the hole in which the delivery system is installed.

Square openings for retainer lugs are provided on both sides of the front cylindrical portion of the body and the walls of the front portion of the body are made thicker within the section between the openings for the retainer lugs. In each square opening two point projections are located on one side at a distance equal to the retainer lug width and designed to hold the retainer lugs in the extreme position and the retainer proper in the closed position, each projection having one flat wall which is directed at an angle to the projection on the side of the retainer open position and along which the retainer lug slides when it changes its position, whereby the force to be applied by the operator's hand to rotate the retainer lugs to close the mechanism is decreased.

The front smooth cylindrical portion of the body has a ring suitably mounted thereon.

The ring has a recess made along the entire internal circumference thereof and designed to receive mating projections located along the circumference of the halves of the rear portion of the body on the side of the retainer so as couple these halves.

On the external circumference of the ring, trapezoidal slots are provided to receive the mating projections of the front end of the rear portion of the body, said projections having atrapezoidal form and designed to fix the rear portion of the body in the ring to prevent its spontaneousrotation while allowing the operator to rotate the rear portion of the body relative to the front portion of the body by the ring.

A cylindrical slot with point elements is made along the circumference of the end face of the smooth cylindrical portion of the front portion of the body on the side of the runner guide, said cylindrical slot designed for coupling with the mating projection with point elements made along the circumference of the internal smooth cylindrical surface of the rear portion of the body on the side of the surface with the double-start thread.

The rear cylindrical portion of the body has longitudinal projections and mating longitudinal slots located on the internal side thereof along the edges of the halves in the zone of the smooth cylindrical surface and used to couple the halves of the rear portion of the body.

Point elements arranged on the internal smooth cylindrical surface of the rear portion of the body along the circumference on the retainer side are designed to reduce friction between the rotatable portions of the body.

A cylindrical projection with point elements is provided along the circumference between the smooth internal surface and the surface with the internal double-start thread of the rear portion of the body to engage the cylindrical slot of the smooth cylindrical part of the front portion of the body on the runner guide side so that the rear cylindrical portion of the body can be rotated about the axis thereof.

Longitudinal projections are made on the external surface of the rear portion of the body, with the projection ends smoothly curved in the opposite directions in order to center the brace fitted over the rear portion of the body and to prevent spontaneous rotation of the brace. Projections to fix the brace in the holes along the circumference of the brace end face are located on the rear end face of the body.

The brace is fitted over the rear portion of the body in the zone of the internal double-start thread.

The brace is essentially a hollow cylinder with internal longitudinal guides along the whole length to ensure snug fit to the external surface of the rear portion of the body on the external side of which mating longitudinal projections are provided having the ends thereof smoothly curved in the opposite directions and used to securely attach the brace.

Holes made along the circumference of the brace end face serve to fix the brace on the surface of the rear portion of the body with the aid of mating projections along the circumference of the end face of the rear portion of the body.

Through holes are made in the front portion of the body, runner, cap, elastic bushing and end face of the rear portion of the body to introduce the coronary stent delivery system or catheter transducer into the device installed in the body.

For easy positioning of the coronary stent on the delivery system or catheter transducer inside the device in the central through hole, chamfers are made on the side of the runner end face and in the rear portion of the body.

The body is made of pressure cast plastic and comprises the front and rear portions, each portion composed of two halves.

Each half of the front and rear portions of the body is solid cast and the two halves are coupled after assembly of the mechanism with the aid of the tapered projections and mating tapered holes made along the internal edges of the front and rear portions of the body and on the transverse walls at the end of the runner guides. The maximum possible number of the tapered projections and mating tapered holes is made on the tapered protrusion of the front portion of the body and in the zone between the openings for the retainer lugs.

The halves of the front portion of the body are coupled with the aid of the tapered projections and mating tapered holes made along the internal edge of the whole body and the tapered projections and mating tapered holes made in the transverse walls at the end of the guides; after the halves of the front portion of the body are coupled, a ring is fitted over the smooth cylindrical portion of the front portion of the body on the side of the guides for the runner, said ring having a recess in which the mating projections of the semicircles of the halves of the rear portion of the body are inserted whereas in the trapezoidal slots made on the external circumference of the ring the mating trapezoidal projections on the semicircles of the front portion of the body are inserted whereupon the halves are coupled so that the smooth cylindrical portion of the front portion of the body is accommodated in the halves of the rear smooth cylindrical portion of the body, with the cylindrical projection with point elements located between the internal smooth portion of the rear portion of the body and the portion having the internal double-start thread being inserted in the cylindrical slot of the smooth cylindrical portion of the front portion of the body on the side of the runner guides and the halves being coupled in the same manner as the front portion of the body with the aid of the tapered projections and mating tapered holes made in the rear portion of the body as well as the longitudinal slots and longitudinal projections on the periphery of the halves of the rear portion of the body thereby forming an overlap joint with the front portion of the body whereupon the cylindrical brace is slipped over the rear portion of the body with the double-start thread on the side of the end face until the brace is fixed on the rear cylindrical portion of the body so that the internal longitudinal guides thereof are positioned between the external longitudinal projections, with the ends of the projections smoothly curved in the opposite directions.

### Embodiment 2 of the Invention

According to this embodiment of the invention the external surface of the rear portion of the body may be smooth without the longitudinal projections having the ends thereof smoothly curved but with the internal longitudinal projections on the internal surface of the brace; the brace in this case is fixed on the rear portion of the body owing to the force provided by surface friction of the parts and brace deformation. The brace in this case should have a diameter allowing the brace to be tightly fitted on the rear portion of the body.

### Embodiment 3 of the Invention

According to this embodiment of the invention the rear end face of the body may have no the projections along the circumference to fix the brace and the brace may have no mating holes and in this case one part is fixed relative to the other part owing to the friction force and tight fit due to brace deformation.

A list of the accompanying drawings follows below.

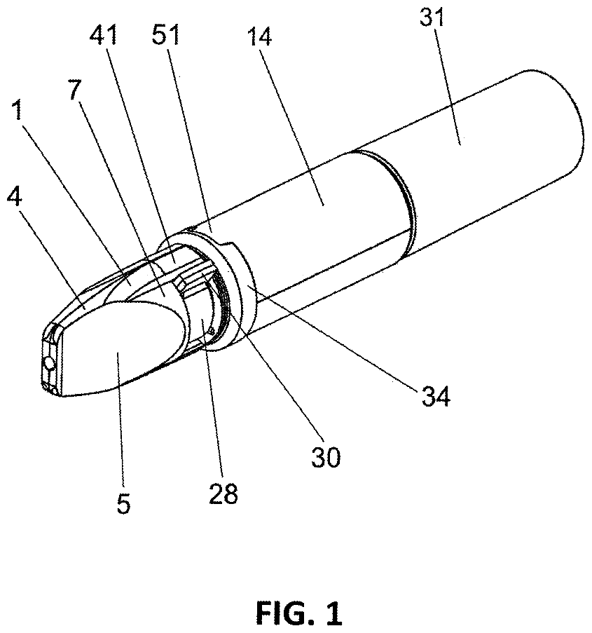

FIG. 1 is a general view of the device body, in which:
1—front portion of the body
4—truncated portion of the front portion of the body
5—recesses for fingers
7—cylindrical portion of the front portion of the body,
14—rear portion of the body
28—retainer
30—retainer lugs
31—brace
34—ring to fix the rear portion of the body
41—thickened wall of the front portion of the body in the zone between the openings for the retainer lugs
51—trapezoidal projection of the rear portion of the body to engage the ring.

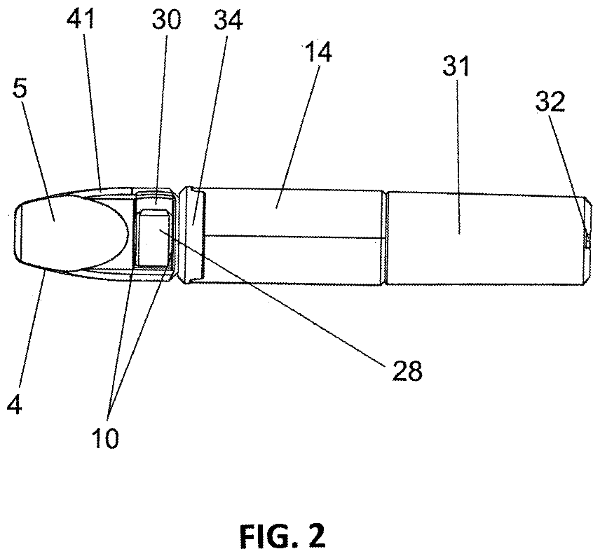

FIG. 2 is a side view of the device body, in which:
4—truncated portion of the front portion of the body
5—recesses for fingers
10—point projection to hold the retainer lugs
14—rear portion of the body
28—retainer
30—retainer lugs
31—brace
32—holes along the circumference of the brace end face for attachment to the rear portion of the body
34—ring to fix the rear portion of the body
41—thickened wall of the front portion of the body in the zone between the openings for the retainer lugs.

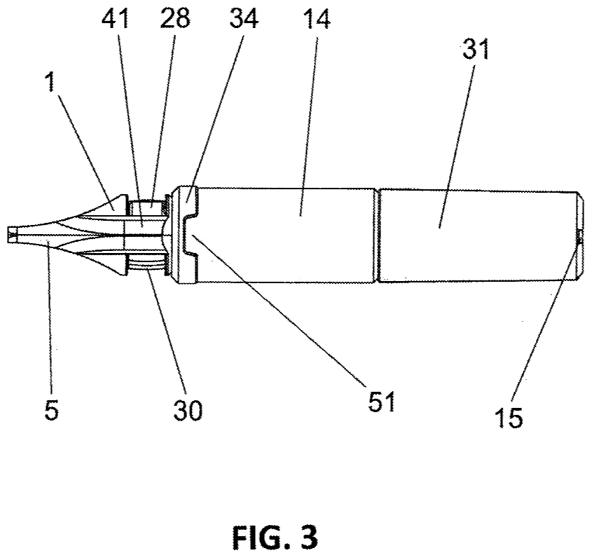

FIG. 3 is a top view of the body, in which:
1—front portion of the body
5—recesses for fingers,
15—projections on the rear end face of the body along the circumference to fix the brace
28—retainer
30—retainer lugs
31—brace
34—ring to fix the rear portion of the body
41—thickened wall of the front portion of the body in the zone between the openings for the retainer lugs
51—trapezoidal projection of the rear portion of the body for fixing in the ring.

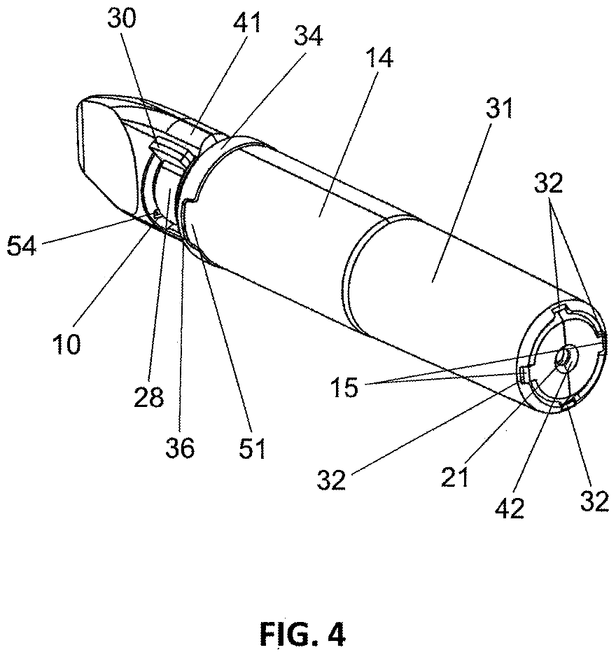

FIG. 4 is a general view of the device body, in which:
10—point projection to hold the retainer lugs
14—rear portion of the body
15—projections on the rear end face of the body along the circumference to fix the brace
21—through hole for the coronary stent delivery system and catheter transducer
28—retainer
30—retainer lugs
31—brace
32—holes along the circumference of the brace end face for attachment to the rear portion of the body
34—ring to fix the rear portion of the body
36—trapezoidal slot of the ring to fix the rear portion of the body
41—thickened wall of the front portion of the body in the zone between the openings for the retainer lugs
42—chamfer with the hole for the delivery system in the rear portion of the body
51—trapezoidal projection of the halves of the rear portion of the body to fix by the ring
54—flat wall of the point projections in the square openings for the retainer lugs.

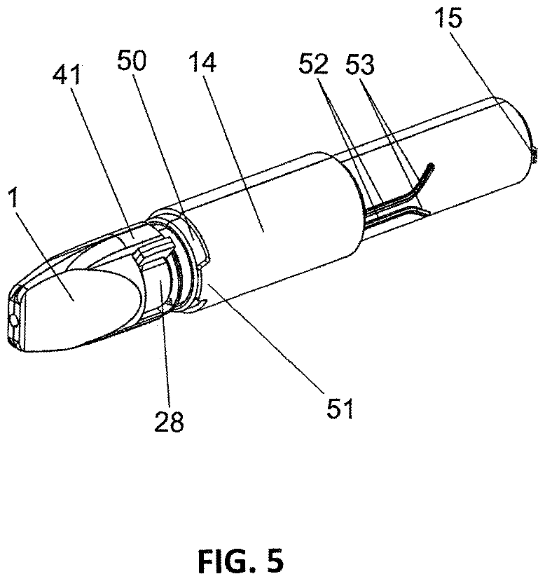

FIG. 5 is a view of the device body without the brace, in which:
1—front portion of the body
14—rear portion of the body
15—projections on the rear end face of the body along the circumference to fix the brace
28—retainer
41—thickened wall of the front portion of the body in the zone between the openings for the retainer lugs
50—projection along the circumference of the halves of the rear portion of the body to couple by the ring
51—trapezoidal projection of the halves of the rear portion of the body to fix by the ring
52—longitudinal projections on the external side of the rear portion of the body to center the brace
53—smoothly curved ends of the longitudinal projection on the external surface of the rear portion of the body.

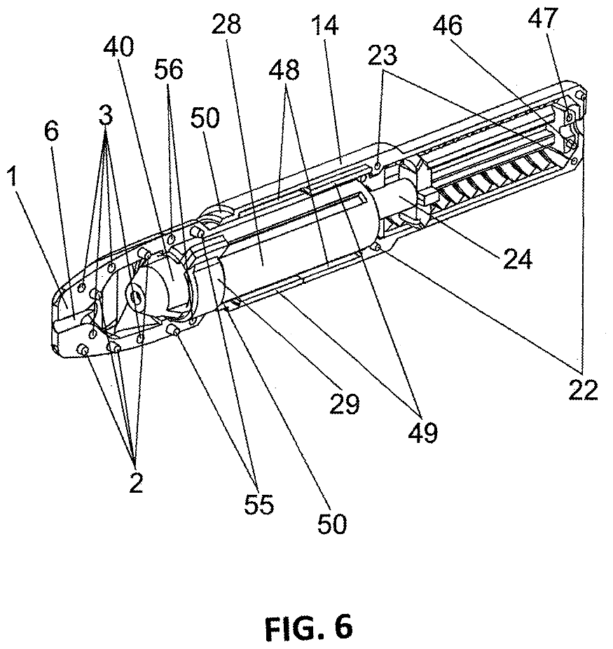

FIG. 6 is a sectional view of the body with the retainer, in which:
1—front portion of the body,
2—tapered projection of the front portion of the body
3—tapered hole of the front portion of the body
6—hole for the delivery system and catheter transducer in the front portion of the body
14—rear portion of the body
22 tapered projection of the rear portion of the body
23—tapered hole of the rear portion of the body
24—runner
28—retainer
29—retainer thickening
40—cap
46—tapered projection of the transversal wall at the guide end
47—tapered hole of the transversal wall at the guide end
48—longitudinal slots of the internal smooth rear portion of the body
49—longitudinal projections of the internal smooth rear portion of the body
50—projection along the circumference of the halves of the rear portion of the body to couple by the ring,
55—tapered projection of the thickened wall of the front portion of the body
56—tapered hole of the thickened wall of the front portion of the body.

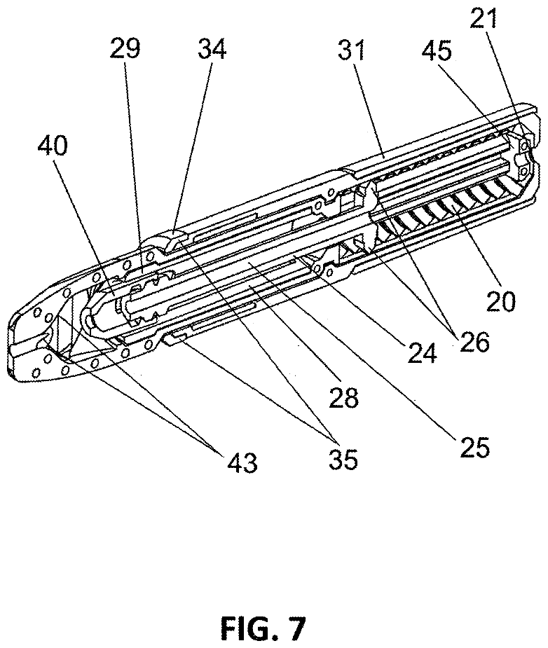

FIG. 7 is a sectional view of the body with the offset thread, in which:
20—internal double-start thread of the rear portion of the body for engagement with the runner
21—through hole for the coronary stent delivery system and catheter transducer in the end face of the body
24—runner
25—through hole in the runner for the coronary stent delivery system and catheter transducer
26—runner projection to engage the thread of the internal surface of the rear portion of the body
28—retainer
29—retainer thickening
31—brace
34—ring to fix the rear portion of the body
35—recess along the internal circumference of the ring to couple the halves of the rear portion of the body
40—cap
43—walls of the tapered hole of the front portion of the body rounded on the internal side
45—transversal wall at the end of the guide of the front portion of the body.

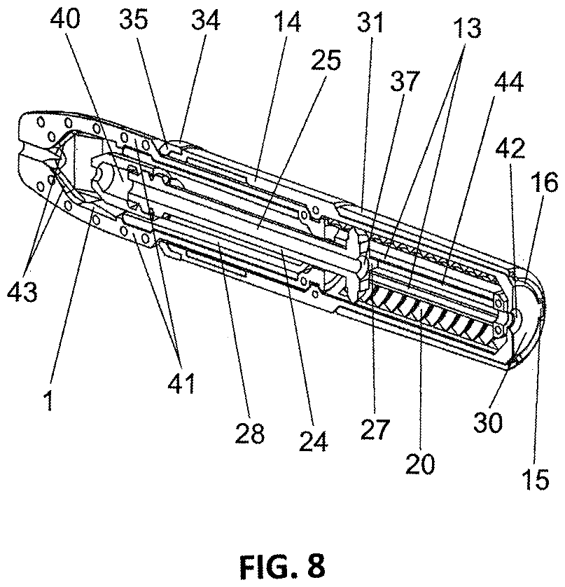

FIG. 8 is a sectional view of the body with the brace, in which:
1—front portion of the body,
13—runner guides
14—rear portion of the body
15—projections to fix the brace
16—chamfer of the rear end face of the body
20—internal double-start thread of the rear portion of the body for engagement with the runner
24—runner
25—hole in the runner for the delivery system or catheter transducer
27—projection for the guides of the front portion of the body
28—retainer
31—cylindrical brace
34—ring to fix the rear portion of the body
35—recess along the internal circumference of the ring to couple the halves of the rear portion of the body,
37—hole for the delivery system and catheter transducer in the runner
38—end face of the rear portion of the body
40—cap
41—thickened wall of the front portion of the body in the zone between the openings for the retainer lugs
42—hole for the delivery system and catheter transducer in the rear portion of the body
43—walls of the tapered hole of the front portion of the body rounded on the internal side.

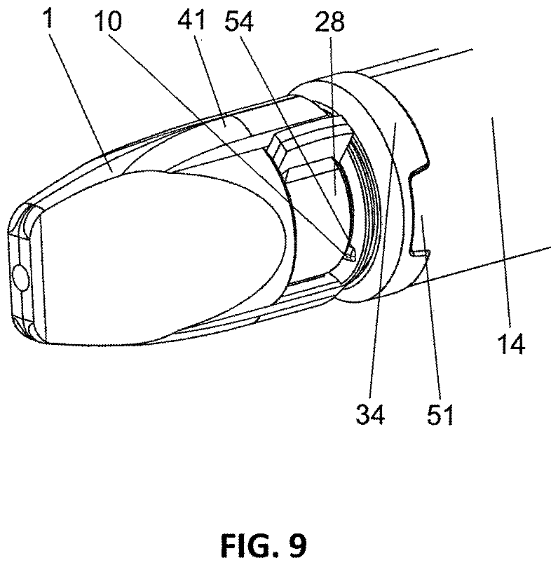

FIG. 9 is a general view of the front portion of the body coupled with the rear portion of the body, in which:
1—front portion of the body
10—point projection to hold the retainer lugs
14—rear portion of the body
28—retainer
34—ring to fix the rear portion of the body
41—thickened wall of the front portion of the body in the zone between the openings for the retainer lugs
51—trapezoidal projection of the halves of the rear portion of the body to fix by the ring
54—flat wall of the point projections in the square openings for the retainer lugs.

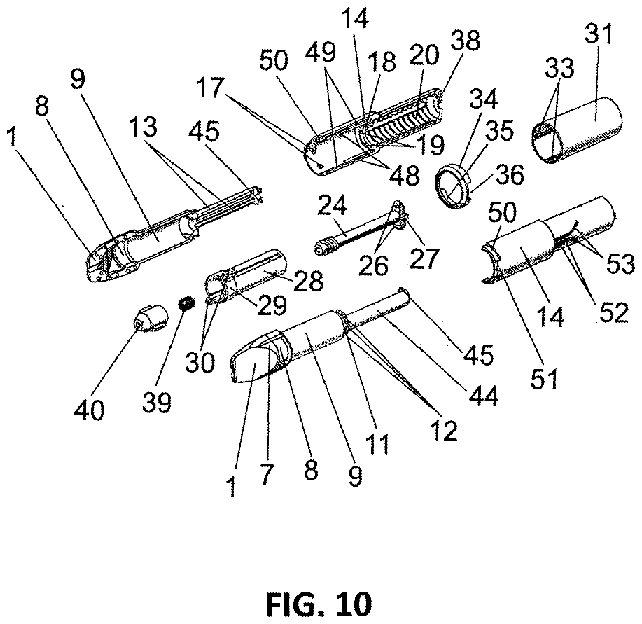

FIG. 10 is an exploded general view of the device, in which:

1—front portion of the body,
7—cylindrical portion of the front portion of the body,
8—openings for the retainer lugs in the front cylindrical portion of the body
9—smooth cylindrical portion of the front portion of the body
11—cylindrical slot of the smooth cylindrical portion of the front portion of the body
12—point elements of the cylindrical slot of the front portion of the body
13—runner guides
14—rear portion of the body
17—point elements of the internal smooth rear portion of the body
18—cylindrical projection
19—point elements of the cylindrical projection
20—internal double-start thread of the rear portion of the body
24—runner
26—runner guides to engage the thread of the internal surface of the rear portion of the body
27—projection for the guides of the front portion of the body
28—retainer
29—retainer thickening
30—retainer lugs
31—brace
33—internal longitudinal projections on the brace
34—ring to fix the rear portion of the body
35—recess along the internal circumference of the ring to couple the halves of the rear portion of the body
36—trapezoidal slot of the ring to fix the rear portion of the body
38—end face of the rear portion of the body
44—longitudinal walls to couple the guides of the front portion of the body
45—transverse walls at the end of the guide of the front portion of the body
48—longitudinal slots of the internal smooth rear portion of the body
49—longitudinal projections of the internal smooth rear portion of the body
50—projection along the circumference of the halves of the rear portion of the body to couple by the ring,
51—trapezoidal projection of the halves of the rear portion of the body to fix by the ring
52—longitudinal projections on the external side of the rear portion of the body to center the brace
53—smoothly curved ends of the longitudinal projection on the external surface of the rear portion of the body.

Now the invention will be described in greater detail with reference to a specific embodiment thereof taken in conjunction with the accompanying drawings.

The body of the device is composed of two components one of which is essentially a part including a front truncated portion 4 (FIG. 1, FIG. 2) on one side thereof and a cylindrical portion 7 (FIG. 1, FIG. 10) on the opposite side thereof, a smooth cylindrical portion 9 (FIG. 10) of a smaller diameter and guides 13 (FIG. 8, FIG. 10) for a runner 24 (FIG. 6, FIG. 7, FIG. 8, FIG. 10) with a chamfer 37 (FIG. 8) with a through hole 21 (FIG. 4, FIG. 7) for a delivery system or a catheter transducer and comprises a front portion 1 (FIG. 1, FIG. 3, FIG. 5, FIG. 6, FIG. 8, FIG. 9, FIG. 10) of the body, and the other component includes a partarranged over the smooth cylindrical portion 9 (FIG. 10) of the first component and a rear part with an internal double-start thread 20 (FIG. 7, FIG. 8, FIG. 10) and a chamfer 16 (FIG. 8) on an end face 38 (FIG. 8) with the through hole 21 (FIG. 4, FIG. 7) for the delivery system or catheter transducer, said second component comprising a rear portion 14 (FIG. 1, FIG. 2, FIG. 3, FIG. 4, FIG. 5, FIG. 6, FIG. 8, FIG. 9, FIG. 10) of the body.

The front portion of the body is the front portion 4 (FIG. 1, FIG. 2) which is truncated on one side and continuously extends on the opposite side in the form of the cylindrical portion 7 (FIG. 1, FIG. 10), the smooth cylindrical portion 9 (FIG. 10) of a smaller diameter and the guides 13 (FIG. 8, FIG. 10) for the runner 24 (FIG. 6, FIG. 7, FIG. 8, FIG. 10) interconnected by longitudinal walls 44 (FIG. 8, FIG. 10) and provided at the end on the end face side of the body with transversal walls 45 (FIG. 7, FIG. 10) having tapered holes 46 (FIG. 6) and mating tapered projections 47 (FIG. 6) to couple the body halves.

The front truncated portion 4 (FIG. 1, FIG. 2) of the body is adapted to be held by fingers of the surgeon's left hand and provided to this end with recesses 5 (FIG. 1, FIG. 2, FIG. 3) allowing the surgeon to fix the device in the desired position, with the recesses 5 (FIG. 1, FIG. 2, FIG. 3) also serving as a pad to fix a coronary catheter. For more convenient arrangement of the coronary stent delivery system or catheter transducer, walls 43 (FIG. 7, FIG. 8) in the front truncated portion 4(FIG. 1, FIG. 2) of the body are made rounded on the internal side opposite to the recesses 5 (FIG. 1, FIG. 2, FIG. 3) for the operator's left hand fingers where said walls contact the mouth of a hole 6 in which the delivery system is installed.

Square openings 8 (FIG. 10) for lugs 30 (FIG. 1, FIG. 2, FIG. 3, FIG. 4, FIG. 10) of a retainer 28 (FIG. 1, FIG. 2, FIG. 3, FIG. 4, FIG. 5, FIG. 6, FIG. 7, FIG. 8, FIG. 9, FIG. 10) are provided on both sides of the front cylindrical portion of the body 1 (FIG. 1, FIG. 3, FIG. 5, FIG. 6, FIG. 8, FIG. 9, FIG. 10) and, in the zone between said holes, walls of the front portion of the body 1 (FIG. 1, FIG. 3, FIG. 5, FIG. 6, FIG. 8, FIG. 9, FIG. 10) near the front truncated portion 4 (FIG. 1, FIG. 2) have a thickening 41 (FIG. 1, FIG. 2, FIG. 3, FIG. 4, FIG. 5, FIG. 8, FIG. 9). In each square opening 8 (FIG. 10) two point projections 10 (FIG. 2, FIG. 4, FIG. 9) are located on one side at a distance equal to the width of the lug 30 (FIG. 1, FIG. 2, FIG. 3, FIG. 4, FIG. 10) of the retainer 28 (FIG. 1, FIG. 2, FIG. 3, FIG. 4, FIG. 5, FIG. 6, FIG. 7, FIG. 8, FIG. 9, FIG. 10), said projections designed to hold the lugs 30 (FIG. 1, FIG. 2, FIG. 3, FIG. 4, FIG. 10) of the retainer 28 (FIG. 1, FIG. 2, FIG. 3, FIG. 4, FIG. 5, FIG. 6, FIG. 7, FIG. 8, FIG. 9, FIG. 10) in the extreme position and the retainer 28 (FIG. 1, FIG. 2, FIG. 3, FIG. 4, FIG. 5, FIG. 6, FIG. 7, FIG. 8, FIG. 9, FIG. 10) proper in the closed position, each projection having one flat wall 54 (FIG. 9), which is directed at an angle to a projection 10 (FIG. 2, FIG. 4, FIG. 9) on the side of the retainer open position and along which the lug 30 (FIG. 1, FIG. 2, FIG. 3, FIG. 4, FIG. 10) of the retainer 28 (FIG. 1, FIG. 2, FIG. 3, FIG. 4, FIG. 5, FIG. 6, FIG. 7, FIG. 8, FIG. 9, FIG. 10) slides when it changes its position, whereby the force to be applied by the operator's hand to rotate the lugs 30 (FIG. 1, FIG. 2, FIG. 3, FIG. 4, FIG. 10) of the retainer 28 (FIG. 1, FIG. 2, FIG. 3, FIG. 4, FIG. 5, FIG. 6, FIG. 7, FIG. 8, FIG. 9, FIG. 10) to close the mechanism is decreased.

The front smooth cylindrical portion 9 (FIG. 10) of the body has a ring 34 (FIG. 1, FIG. 2, FIG. 3, FIG. 4, FIG. 7, FIG. 8, FIG. 9, FIG. 10) suitably mounted thereon.

The ring 34 (FIG. 1, FIG. 2, FIG. 3, FIG. 4, FIG. 7, FIG. 8, FIG. 9, FIG. 10) has a recess 35 (FIG. 7, FIG. 8, FIG. 10) made along the entire internal circumference thereof and designed to receive mating projections 50 (FIG. 5, FIG. 6, FIG. 10) located along the circumference of the halves of the rear portion 14 (FIG. 1, FIG. 2, FIG. 3, FIG. 4, FIG. 5, FIG. 6, FIG. 8, FIG. 9, FIG. 10) of the body on the side of the retainer 28 (FIG. 1, FIG. 2, FIG. 3, FIG. 4, FIG. 5, FIG. 6, FIG. 7, FIG. 8, FIG. 9, FIG. 10) so as couple these halves.

On the external circumference of the ring 34 (FIG. 1, FIG. 2, FIG. 3, FIG. 4, FIG. 7, FIG. 8, FIG. 9, FIG. 10), trapezoidal slots 36 (FIG. 4, FIG. 10) are provided to receive the mating projections 51 (FIG. 1, FIG. 3, FIG. 4, FIG. 5, FIG. 9, FIG. 10) of the front end of the rear portion 14 (FIG. 1, FIG. 2, FIG. 3, FIG. 4, FIG. 5, FIG. 6, FIG. 8, FIG. 9, FIG. 10) of the body, said projections having a trapezoidal form and designed to fix the rear portion 14 (FIG. 1, FIG. 2, FIG. 3, FIG. 4, FIG. 5, FIG. 6, FIG. 8, FIG. 9, FIG. 10) of the body in the ring 34 (FIG. 1, FIG. 2, FIG. 3, FIG. 4, FIG. 7, FIG. 8, FIG. 9, FIG. 10) to prevent its spontaneous rotation.

A cylindrical slot 11 (FIG. 10) with point elements 12 (FIG. 10) is provided along the circumference of the end face of the smooth cylindrical portion of the front portion 9 (FIG. 10) of the body on the side of the guide 13 (FIG. 8, FIG. 10) of the runner 24 (FIG. 6, FIG. 7, FIG. 8, FIG. 10), said cylindrical slot designed for coupling with a mating projection 18 (FIG. 10) with point elements 19 (FIG. 10) made along the circumference of the internal smooth cylindrical surface of the rear portion 14 (FIG. 1, FIG. 2, FIG. 3, FIG. 4, FIG. 5, FIG. 6, FIG. 8, FIG. 9, FIG. 10) of the body on the side of the surface with the double-start thread 20 (FIG. 7, FIG. 8, FIG. 10).

The rear cylindrical portion 14 (FIG. 1, FIG. 2, FIG. 3, FIG. 4, FIG. 5, FIG. 6, FIG. 8, FIG. 9, FIG. 10) of the body has longitudinal projections 49 (FIG. 6, FIG. 9) and mating longitudinal slots 48 (FIG. 6, FIG. 9) located on the internal side thereof along the edges of the halves in the smooth cylindrical surface zone and used to couple the halves of the rear portion 14 (FIG. 1, FIG. 2, FIG. 3, FIG. 4, FIG. 5, FIG. 6, FIG. 8, FIG. 9, FIG. 10) of the body.

Point elements 17 (FIG. 10) arranged on the internal smooth cylindrical surface of the rear portion 14 (FIG. 1, FIG. 2, FIG. 3, FIG. 4, FIG. 5, FIG. 6, FIG. 8, FIG. 9, FIG. 10) of the body along the circumference on the retainer side are designed to reduce friction between the rotatable portions of the body.

The cylindrical projection 18 (FIG. 10) with the point elements 19 (FIG. 10) is provided along the circumference between the smooth internal surface and the surface with the internal double-start thread 20 (FIG. 7, FIG. 8, FIG. 10) of the rear portion 14 (FIG. 1, FIG. 2, FIG. 3, FIG. 4, FIG. 5, FIG. 6, FIG. 8, FIG. 9, FIG. 10) of the body to engage the cylindrical slot 11 (FIG. 10) of the smooth cylindrical part of the front portion 9 (FIG. 10) of the body on the side of the guide 13 (FIG. 8, FIG. 10) of the runner 24 (FIG. 6, FIG. 7, FIG. 8, FIG. 10) so that the rear cylindrical portion 14 (FIG. 1, FIG. 2, FIG. 3, FIG. 4, FIG. 5, FIG. 6, FIG. 8, FIG. 9, FIG. 10) of the body can be rotated about the axis thereof.

Longitudinal projections 52 (FIG. 5, FIG. 10) are made on the external surface of the rear portion 14 (FIG. 1, FIG. 2, FIG. 3, FIG. 4, FIG. 5, FIG. 6, FIG. 8, FIG. 9, FIG. 10) of the body, with ends 53 (FIG. 5, FIG. 10) smoothly curved in the opposite directions in order to center a brace 31 (FIG. 1, FIG. 2, FIG. 3, FIG. 4, FIG. 7, FIG. 8, FIG. 10) fitted over the rear portion 14 (FIG. 1, FIG. 2, FIG. 3, FIG. 4, FIG. 5, FIG. 6, FIG. 8, FIG. 9, FIG. 10) of the body and to prevent spontaneous rotation of the brace. Projections 15 (FIG. 3, FIG. 4, FIG. 5, FIG. 8) to fix the brace 31 (FIG. 1, FIG. 2, FIG. 3, FIG. 4, FIG. 7, FIG. 8, FIG. 10) in holes 32 (FIG. 3, FIG. 4) along the circumference of the end face of the brace 31 (FIG. 1, FIG. 2, FIG. 3, FIG. 4, FIG. 7, FIG. 8, FIG. 10) are located on the end face 38 (FIG. 8) of the rear portion 14 (FIG. 1, FIG. 2, FIG. 3, FIG. 4, FIG. 5, FIG. 6, FIG. 8, FIG. 9, FIG. 10) of the body.

The brace 31 (FIG. 1, FIG. 2, FIG. 3, FIG. 4, FIG. 7, FIG. 8, FIG. 10) is fitted over the rear portion 14 (FIG. 1, FIG. 2, FIG. 3, FIG. 4, FIG. 5, FIG. 6, FIG. 8, FIG. 9, FIG. 10) of the body in the zone of the internal double-start thread 20 (FIG. 7, FIG. 8, FIG. 10).

The brace 31 (FIG. 1, FIG. 2, FIG. 3, FIG. 4, FIG. 7, FIG. 8, FIG. 10) is essentially a hollow cylinder with internal longitudinal guides 33 along the whole length to ensure snug fit to the external surface of the rear portion 14 (FIG. 1, FIG. 2, FIG. 3, FIG. 4, FIG. 5, FIG. 6, FIG. 8, FIG. 9, FIG. 10) of the body on the external side of which the mating longitudinal projections 52 (FIG. 5, FIG. 10) are provided with the ends 53 (FIG. 5, FIG. 10) thereof smoothly curved in the opposite directions, said projections used to securely attach the brace (FIG. 1, FIG. 2, FIG. 3, FIG. 4, FIG. 7, FIG. 8, FIG. 10).

The holes 32 (FIG. 2, FIG. 4) made along the circumference of the end face of the brace 31 (FIG. 1, FIG. 2, FIG. 3, FIG. 4, FIG. 7, FIG. 8, FIG. 10) serve to fix the brace 31 (FIG. 1, FIG. 2, FIG. 3, FIG. 4, FIG. 7, FIG. 8, FIG. 10) on the surface of the rear portion 14 (FIG. 1, FIG. 2, FIG. 3, FIG. 4, FIG. 5, FIG. 6, FIG. 8, FIG. 9, FIG. 10) of the body with the aid of mating projections 15 (FIG. 3, FIG. 4, FIG. 5, FIG. 8) along the circumference of the end face 38 (FIG. 8) of the rear portion 14 (FIG. 1, FIG. 2, FIG. 3, FIG. 4, FIG. 5, FIG. 6, FIG. 8, FIG. 9, FIG. 10) of the body.

Through holes 6 (FIG. 6), 21 (FIG. 4, FIG. 7) and 25 (FIG. 7, FIG. 8) are made in the front portion 1 (FIG. 1, FIG. 3, FIG. 5, FIG. 6, FIG. 8, FIG. 9, FIG. 10) of the body, the end face 38 of the rear portion 14 (FIG. 1, FIG. 2, FIG. 3, FIG. 4. FIG. 5, FIG. 6, FIG. 8, FIG. 9, FIG. 10) of the body, the runner 24 (FIG. 6, FIG. 7, FIG. 8, FIG. 10), a cap 40 (FIG. 6, FIG. 7, FIG. 8, FIG. 10) and an elastic bushing 39 (FIG. 10) to introduce the coronary stent delivery system or pull catheter transducer into the device installed in the body.

For easy positioning of the coronary stent on the delivery system or catheter transducer inside the device in the central through hole 6 (FIG. 6), the central hole 21 (FIG. 4, FIG. 7) and a central hole 25 (FIG. 7, FIG. 8), a chamfer 42 (FIG. 8) with the hole 21 (FIG. 7) is made on the end face 38 (FIG. 8) of the rear portion 14 (FIG. 1, FIG. 2, FIG. 3, FIG. 4, FIG. 5, FIG. 6, FIG. 8, FIG. 9, FIG. 10) of the body whereas the chamfer 37 (FIG. 8) with the hole 25 (FIG. 7) is made on the end face of the runner 24 (FIG. 6, FIG. 7 FIG. 8, FIG. 10).

The body comprises the front portion 1 (FIG. 1, FIG. 3, FIG. 5, FIG. 6, FIG. 8, FIG. 9, FIG. 10) and the rear portion 14 (FIG. 1, FIG. 2, FIG. 3, FIG. 4, FIG. 5, FIG. 6, FIG. 8, FIG. 9, FIG. 10) made of pressure cast plastic and is composed of two identical halves which are coupled after the mechanism is assembled.

The front portion of the body is assembled with the aid of tapered projections 2 (FIG. 6), 55 (FIG. 6) and mating tapered holes 3 (FIG. 6), 56 (FIG. 6) made along the internal edges of the front portion 1 (FIG. 1, FIG. 3, FIG. 5, FIG. 6, FIG. 8, FIG. 9, FIG. 10) of the body, with the maximum possible number of the tapered projections 3 (FIG. 6) and the mating tapered holes 2 (FIG. 6) made on the tapered projection 22 (FIG. 6) of the front portion 1 (FIG. 1, FIG. 3, FIG. 5, FIG. 6, FIG. 8, FIG. 9, FIG. 10) of the body, tapered projections 55 (FIG. 6) and the mating tapered holes 56 (FIG. 6) in the zone between the lugs 30 (FIG. 1, FIG. 2, FIG. 3, FIG. 4, FIG. 10) of the retainer 28 (FIG. 1, FIG. 2, FIG. 3, FIG. 4, FIG. 5, FIG. 6, FIG. 7, FIG. 8, FIG. 9, FIG. 10) and the tapered projections 46 (FIG. 6) with the mating tapered holes 47 (FIG. 6) on the transversal walls (FIG. 7, FIG. 10) at the end of the guides 13 (FIG. 8) of the runner 24 (FIG. 6, FIG. 7, FIG. 8, FIG. 10) of the front portion 1 (FIG. 6) of the body, whereby jointing reliability is made higher.

After the halves of the front portion 1 (FIG. 1, FIG. 3, FIG. 5, FIG. 6, FIG. 8, FIG. 9, FIG. 10) of the body are coupled, the ring 34 (FIG. 1, FIG. 2, FIG. 3, FIG. 4, FIG. 7, FIG. 8, FIG. 9, FIG. 10) is fitted over the smooth cylindrical portion 9 (FIG. 10) of the front portion of the body on the side of the guides 13 (FIG. 8) for the runner 24 (FIG. 6, FIG. 7, FIG. 8, FIG. 10), said ring having the recess 35 (FIG. 7, FIG. 8, FIG. 10) in which the mating projections 50 (FIG. 6, FIG. 10) of the semicircles of the halves of the rear portion 14 (FIG. 1, FIG. 2, FIG. 3, FIG. 4, FIG. 5, FIG. 6, FIG. 8, FIG. 9, FIG. 10) of the body are inserted whereas in the trapezoidal slots 36 (FIG. 10) made on the external circumference of the ring 34 (FIG. 2, FIG. 3, FIG. 4, FIG. 7, FIG. 8, FIG. 9, FIG. 10) the mating trapezoidal projections 51 (FIG. 1, FIG. 3, FIG. 4, FIG. 5, FIG. 9, FIG. 10) on the semicircles of the halves of the front portion of the rear portion 14 (FIG. 1, FIG. 2, FIG. 3, FIG. 4, FIG. 5, FIG. 6, FIG. 8, FIG. 9, FIG. 10) of the body are inserted whereupon the halves are coupled so that the smooth cylindrical portion of the front portion 9 (FIG. 10) of the body is accommodated in the halves of the rear smooth cylindrical portion 14 (FIG. 1, FIG. 2, FIG. 3, FIG. 4, FIG. 5, FIG. 6, FIG. 8, FIG. 9, FIG. 10) of the body, wherein the cylindrical projection 18 (FIG. 10) and the point elements 19 (FIG. 10) located between the internal smooth portion of the rear portion 14 (FIG. 10) of the body and the portion having the internal double-start thread 20 (FIG. 10) are inserted in the cylindrical slot 11 (FIG. 10) with the point elements 12 (FIG. 10) of the smooth cylindrical portion of the front portion 9 (FIG. 10) of the body on the side of the runner guides and the halves are coupled in the same manner as the front portion of the body with the aid of the tapered projections 22 (FIG. 6) and mating tapered holes 23 (FIG. 6) made in the rear portion 14 (FIG. 1, FIG. 2, FIG. 3, FIG. 4, FIG. 5, FIG. 6, FIG. 8, FIG. 9, FIG. 10) of the body and with the aid of the longitudinal slots 48 (FIG. 6, FIG. 10) and the longitudinal projections 49 (FIG. 6, FIG. 10) along the edges of the halves of the rear internal smooth part of the rear portion of the body thereby forming an overlap joint with the front portion 1 (FIG. 1, FIG. 3, FIG. 5, FIG. 6, FIG. 8, FIG. 9, FIG. 10) of the body whereupon the cylindrical brace 31 (FIG. 1, FIG. 2, FIG. 3, FIG. 4, FIG. 7, FIG. 8, FIG. 10) is slipped over the rear portion 14 (FIG. 1, FIG. 2, FIG. 3, FIG. 4, FIG. 5, FIG. 6, FIG. 8, FIG. 9, FIG. 10) of the body in the zone of the internal double-start thread 20 (FIG. 7, FIG. 8, FIG. 10) on the side of the end face 38 (FIG. 8) until said brace is fixed on the rear cylindrical portion 14 (FIG. 1, FIG. 2, FIG. 3, FIG. 4, FIG. 5, FIG. 6, FIG. 8, FIG. 9, FIG. 10) of the body so that the internal longitudinal guides 33 (FIG. 10) thereof are positioned between the external longitudinal projections 52 (FIG. 5, FIG. 10) of the rear portion of the body, with the ends 53 (FIG. 5, FIG. 10) of said projections smoothly curved in the opposite directions.

### Embodiment 2 of the Invention

According to this embodiment of the invention the external surface of the rear portion 14 (FIG. 1, FIG. 2, FIG. 3, FIG. 4, FIG. 5, FIG. 6, FIG. 8, FIG. 9, FIG. 10) of the body may be smooth without the external longitudinal projections 52 (FIG. 5, FIG. 10) having the ends 53 (FIG. 5, FIG. 10) thereof smoothly curved but with the internal longitudinal projections 33 (FIG. 10) on the internal surface of the brace 31 (FIG. 10); the brace 31 (FIG. 1, FIG. 2, FIG. 3, FIG. 4, FIG. 7, FIG. 8, FIG. 10) in this case is fixed on the rear portion 14 (FIG. 1, FIG. 2, FIG. 3, FIG. 4, FIG. 5, FIG. 6, FIG. 8, FIG. 9, FIG. 10) of the body owing to the force provided by surface friction of the parts and brace deformation. The brace 31 (FIG. 1, FIG. 2, FIG. 3, FIG. 4, FIG. 7, FIG. 8, FIG. 10) in this case should have a diameter allowing the brace to be tightly fitted on the rear portion 14 (FIG. 1, FIG. 2, FIG. 3, FIG. 4, FIG. 7, FIG. 8, FIG. 9, FIG. 10) of the body.

### Embodiment 3 of the Invention

According to this embodiment of the invention the rear end face 38 (FIG. 8) of the body may have no projections 15 (FIG. 3, FIG. 8) along the circumference to fix the brace 31 (FIG. 1, FIG. 2, FIG. 3, FIG. 4, FIG. 7, FIG. 8, FIG. 10) and the brace 31 (FIG. 1, FIG. 2, FIG. 3, FIG. 4, FIG. 7, FIG. 8, FIG. 10) may have no mating holes 32 (FIG. 2, FIG. 4) to receive said projections 15 (FIG. 3, FIG. 8) and in this case one part is fixed relative to the other part owing to the friction force and tight fit provided by deformation of the brace 31 (FIG. 1, FIG. 2, FIG. 3, FIG. 4, FIG. 7, FIG. 8, FIG. 10).

1. A body of the device suitable for positioning a coronary stent in coronary arteries or pulling a catheter transducer for intravascular ultrasound, said body comprising a retainer with lugs, a runner with an opening to accommodate a delivery system therein and a chamfered rear portion, said runner provided with projections to engage an internal thread of a rear cylindrical portion of the body and projections for movement along guides inside the body on one side and with a cap with a hole having an elastic bushing with an internal hole with tapered and end surfaces conforming to surfaces inside the cap and the runner, said bushing positioned in the threaded cap to be screwed on the runner on the opposite side, wherein the body is composed of the front and rear portions made of two halves; the front portion of the body is truncated on one side thereof and continuously extends on the opposite side thereof in the form of a cylindrical portion with openings for retainer lugs with point projections in each opening, smooth cylindrical portion of a smaller diameter and runner guides with a cylindrical slot with point elements in front of the guides, whereas the rear portion of the body is made cylindrical and has an internal smooth surface, having point elements along the circumference thereof, and a surface with a double-start thread along the circumference thereof, wherein a cylindrical projection with a point element is provided between the smooth internal surface and the surface with the double-start thread along the circumference thereof, the end face od the rear portion has a chamfer and a through hole is made in the front portion and the end face of the rear portion to introduce the coronary stent delivery system or the catheter transducer for intravascular ultrasound into the device interior,
characterized in that said body is composed of:
the front portion of the body comprising:
rounded walls in the truncated front portion of the body on the internal side opposite to recesses for the fingers of the operator's left hand in a place where said walls adjoin the mouth of the input hole to accommodate the delivery system or the catheter transducer,
thickened walls in the zone between the opening for the retainer lugs of the front portion of the body,
flat walls located at an angle to the point projections in the openings the for the retainer lugs,
a ring rotatably mounted on the smooth cylindrical surface of the front portion of the body having a recess in which projections of halves of the rear portion of the body are located along the circumference to couple said halves of the rear portion of the body whereas in trapezoidal slots trapezoidal projections of the halves of the rear portion of the body are located to fix said halves of the rear portion of the body,
longitudinal walls connecting the runner guides and transversal walls adjoining at the end of the guides are provided with a tapered projection and a mating tapered hole and the chamfered runner near a central hole,
the rear portion of the body comprising:
longitudinal projections and mating slots along the edges of the internal smooth portion of the halves of the body to couple said halves of the body,
longitudinal projections having the ends thereof curved in the opposite directions on the external surface of the body in the zone with the internal double-start thread to engage the longitudinal projections along the circumference of the internal surface of a brace,
the brace having the form of a hollow cylinder and provided with:
longitudinal projections along the circumference of the internal surface to engage the longitudinal projections with having the ends curved in the opposite directions and located on the external surface in the zone of the rear portion of the body with the internal double-start thread, holes in the end face for attachment to the end face of the rear portion of the body for attachment in the projections along the circumference of the end face of the rear portion of the body to hold the brace.

2. The body of the device as claimed in claim 1, wherein the external surface of the rear portion of said body is smooth and has no longitudinal projections smoothly curved in the opposite directions on the external surface.

3. The body of the device as claimed in claim 1, wherein the end face of the cylindrical brace has no holes along the circumference thereof whereas the end face of the rear portion of the body has no mating projections along the circumference thereof.
